(19) United States
(12) Patent Application Publication
(10) Pub. No.: US 2021/0220583 A1
(43) Pub. Date: Jul. 22, 2021

(54) INHALATOR

(71) Applicants: Denis Aleksandrovich Gorbatyuk, St.Petersburg (RU); Anton Vladimirovich Frolov, Bell Canyon, CA (US)

(72) Inventors: Denis Aleksandrovich Gorbatyuk, St.Petersburg (RU); Anton Vladimirovich Frolov, Bell Canyon, CA (US)

(21) Appl. No.: 17/214,831

(22) Filed: Mar. 27, 2021

Related U.S. Application Data
(63) Continuation-in-part of application No. PCT/RU2019/000689, filed on Sep. 27, 2019.

(30) Foreign Application Priority Data
Sep. 27, 2018 (RU) 2018134112

(51) Int. Cl. A61M 15/08; A61M 15/00

(52) U.S. Cl. CPC A61M 15/085; A61M 2202/0057; A61M 2202/0007; A61M 15/0086

(57) ABSTRACT

An inhalation device and a method for providing an inhalation device are described herein. An inhalation device includes a flat element having an open ring shape. The flat element has end parts and a gap provided between the end parts to configure the flat element to be held on a nose. The flat element has a horizontal plane disposed perpendicularly to a symmetry plane of the flat element. The flat element includes at least two layers. The first layer includes a substrate and a second layer includes at least one layer of a material impregnated with one or more volatile substances. The at least one layer of the material impregnated with the one or more volatile substances is fixed on the substrate.

### CROSS-REFERENCE TO RELATED APPLICATIONS

This application is a Continuation-in-part and claims priority of International Application No. PCT/RU2019/000689, filed on Sep. 27, 2019, entitled “INHALATOR” which claims priority of Russian Patent Application No. 2018/134112 filed on Sep. 27, 2018, entitled “INHALATOR.”. The subject matter of aforementioned applications is incorporated herein by reference in its entirety.

### TECHNICAL FIELD

The present disclosure relates generally to health care devices and, more specifically, to inhalation devices for performing cold inhalation procedures by releasing volatile aromatic or medicinal substances into the air to provide a positive effect on patients. The inhalation devices can be used at health resorts, in medical practice, or in everyday life for health or medical procedures.

### BACKGROUND

The approaches described in this section could be pursued but are not necessarily approaches that have previously been conceived or pursued. Therefore, unless otherwise indicated, it should not be assumed that any of the approaches described in this section qualify as prior art merely by virtue of their inclusion in this section.

Inhalation is a method of administering medicinal products into respiratory channels of a patient by inhaling particles of the medicinal products by the patient. Types of inhalation include a hot inhalation when a wormed product is inhaled and a cold inhalation when a product inhaled by the patient has a room temperature. The hot inhalation is usually used when the medicinal product is not volatile under the room temperature, while volatile substances are usually used for the cold inhalation.

Conventional devices for cold inhalation are usually placed close to or inserted into a nose of a patient. However, upon being placed into the nose, a part of the device filled with volatile substances may contact a skin or a mucous membrane of the patient and may became contaminated, as well as may cause a risk of skin damage and allergic reactions due to attachment directly to the skin or the mucous membrane.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Provided are an inhalation device and a method for providing an inhalation device. In some example embodiments, an inhalation device may include a flat element having an open ring shape. The flat element may have end parts and a gap provided between the end parts to configure the flat element to be held on a nose. The flat element may have a horizontal plane disposed perpendicularly to a symmetry plane of the flat element. The flat element may include at least two layers. The first layer may include a substrate and a second layer may include at least one layer of a material impregnated with one or more volatile substances. The at least one layer of the material impregnated with the one or more volatile substances may be fixed on the substrate.

A method for providing an inhalation device may commence with providing a flat element having a ring shape. The flat element may include a substrate. The method may further include applying at least one layer of an adsorbent material onto the substrate. The method may continue with impregnating the at least one layer of the adsorbent material with one or more volatile substances. The method may further include cutting the flat element to provide an open ring shape of the flat element and provide a gap between end parts of the flat element to configure the flat element to be held on a nose.

Additional objects, advantages, and novel features will be set forth in part in the detailed description section of this disclosure, which follows, and in part will become apparent to those skilled in the art upon examination of this specification and the accompanying drawings or may be learned by production or operation of the example embodiments. The objects and advantages of the concepts may be realized and attained by means of the methodologies, instrumentalities, and combinations particularly pointed out in the appended claims.

### BRIEF DESCRIPTION OF THE DRAWINGS

Embodiments are illustrated by way of example and not limitation in the figures of the accompanying drawings, in which like references indicate similar elements and in which:

FIG. 1 is a general view of an inhalation device, in accordance with some embodiments.

FIG. 2 is a cross-sectional view of an inhalation device, in accordance with certain embodiments.

FIG. 3 is a flow chart illustrating a method for providing an inhalation device, in accordance with an example embodiment.

### DETAILED DESCRIPTION

The following detailed description includes references to the accompanying drawings, which form a part of the detailed description. The drawings show illustrations in accordance with exemplary embodiments. These exemplary embodiments, which are also referred to herein as “examples,” are described in enough detail to enable those skilled in the art to practice the present subject matter. The embodiments can be combined, other embodiments can be utilized, or structural, logical, and electrical changes can be made without departing from the scope of what is claimed. The following detailed description is, therefore, not to be taken in a limiting sense, and the scope is defined by the appended claims and their equivalents.

The present disclosure provides an inhalation device and a method for providing an inhalation device. The inhalation device (also referred herein to an inhalator) may include a flat element having a shape of an open ring. The flat element may have end parts near an open portion of the open ring and a gap provided between the end parts. The gap may configure the flat element to be held in a nose of a patient. Specifically, due the end parts and the gap between the end parts, the flat element may be configured for placing on a septum or wings of the nose.

In example embodiments, the open ring shape of the flat element may include any of a horseshoe shape, a round open ring shape, an oval open ring shape, an ellipsoid open ring shape, a U-shape, and any similar shape. The flat element may have a horizontal plane (also referred to a plane of the flat element) disposed perpendicularly to a symmetry plane of the flat element.

The flat element may have at least two layers. The two layers may include a substrate and at least one layer of a material impregnated with volatile substances. The layer of the material impregnated with the volatile substances may be placed directly onto the substrate and fastened to the substrate by pressing. In some embodiments, an adhesive layer may be placed between the substrate and the layer of the material impregnated with volatile substances to attach the layer of the material impregnated with volatile substances to the substrate.

The flat element may be an element having a comparatively small thickness. Specifically, the thickness of the flat element may be less than the length of the flat element. An upper and a lower surface of the flat element can be flat or can be slightly rounded in the course of technological operations during manufacturing of the flat element.

The end parts of the flat element may be configured to act as means for retention of the flat element on the septum or wings of the nose. The layer of the material impregnated with volatile substances may be attached to an upper surface of the substrate. When a patient uses the inhalation device, the material impregnated with volatile substances does not contact a skin or a mucous membrane of the patient. Therefore, the layer with the material impregnated with volatile substances is prevented from contamination with particles of the skin or body fluids of the patient. Additionally, as the layer with the material impregnated volatile substances of the inhalation device does not contact the skin, the use of the inhalation device avoids the risk of skin damage and allergic reactions. Therefore, the structure of the inhalation device of the present disclosure makes the inhalation device convenient and safe to use, as well reduces the probability of allergic reactions and skin damage, provides conditions for compliance with hygienic requirements when using the inhalation device, and expands the range of applicable compositions of volatile substances. Additionally, the inhalation device of the present disclosure is a ready-to-use device and eliminates the discomfort associated with the need for the patient to prepare a conventional inhalator for use, e.g., by removing protective covers or attaching a layer with working substances (such as volatile substances) to a body of the inhalator.

The structure of the inhalation device ensures compliance with hygienic requirements when using the inhalation device. Specifically, when the inhalation device is used by a patient, the inhalation device is facing nasal passages by a lateral surface of the flat element, which reduces the area of possible contamination of the inhalation device with exhaled substances, while a working plane of the flat element impregnated with volatile substances is located along the inhaled/exhaled stream of air. Moreover, the material used in the layer impregnated with one or more volatile substances may be unreactive to volatile substances, therefore, a wide range of volatile substances (aromatic and medicinal) may be used for the impregnation of the layer placed on the substrate.

In an example embodiment, the substrate of the inhalation device may be made of paper or cardboard. The layer of the material impregnated with the volatile substances may be made of a porous material or a material having a fiber structure. In a further example embodiment, the layer of the material impregnated with the volatile substances may be fixed to the substrate directly (e.g., by pressing) or by using an adhesive layer placed between the substrate and the layer of the material impregnated with the volatile substances. In a further example embodiment, the material for the layer impregnated with the volatile substances may be selected from the following materials: a porous artificially synthesized polyurethane, cork tree, cotton, linen, paper, cardboard, wood veneer, silk, wool, felt, or combinations of these materials. An impregnating compound used for impregnating the material may include essential oils, essential compositions, and/or medicinal substances.

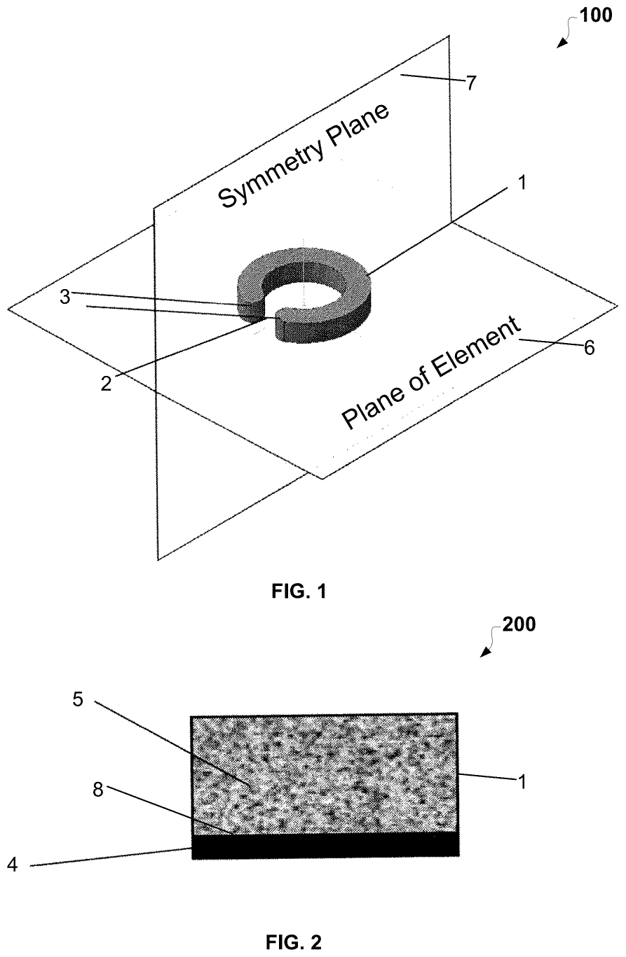

Referring now to the drawings, FIG. 1 illustrates a general view of an inhalation device 100, according to an example embodiment. The inhalation device 100 may include a flat element 1 having an open ring shape with a gap 2 between end parts 3 of the flat element 1. The gap 2 and the end parts 3 may configure the flat element 1 to be held on a nose of a patient. In an example embodiment, the open ring shape may include one of the following shapes: a horseshoe shape, a round open ring shape, an oval open ring shape, an ellipsoid open ring shape, a U-shape, and so forth. Holding of the flat element 1 on the nose may include holding of the flat element on a septum or wings of the nose. The flat element 1 may have a horizontal plane (shown as a plane 6 of the element) disposed perpendicularly to a symmetry plane 7 of the flat element 1.

FIG. 2 is a cross-sectional view 200 of the inhalation device 100, according to an example embodiment. The flat element 1 may include at least two layers. Specifically, the flat element 1 may include a substrate 4 and at least one layer 5 of a material impregnated with volatile substances. The at least one layer 5 of the material impregnated with volatile substances may be fixed to the substrate 4. FIG. 2 shows one layer 5 of the material impregnated with volatile substances, but any number of layers 5 of the material impregnated with volatile substances may be applied on the substrate 4 in example embodiments of the inhalation device 100.

In an example embodiment, the substrate 4 may be made of paper, cardboard, or any other material. The layer 5 of the material impregnated with the volatile substances may be made of a porous material, a material having a fiber structure, or any material applicable for being impregnated with volatile substances. In particular, the material (also referred herein to as an adsorbent material) of layer 5 applicable for being impregnated with the volatile substances may be selected from the following materials: a porous polyurethane, cork tree, cotton, linen, paper, cardboard, wood veneer, silk, wool, felt, and any combinations of these materials. The volatile substances may include at least one of essential oils, essential compositions, medicinal substances, and so forth. The volatile substances may be selected from natural materials to allow complete evaporation of the volatile substances, including all fractions of the volatile substances. The inhalation device 100 can be placed into a closed sealed package.

To use the inhalation device 100, a patient may place the inhalation device 100 on a septum of a nose of the patient. It is also possible to place the inhalation device 100 on a wing of the nose. The gap 2 between the end parts 3 of the flat element 1 may correspond to a regular size of a septum or a wing of a nose. The end parts 3 may be rounded to provide roundings at the end parts 3 of the flat element 1 and ensure comfortable placement of the inhalation device 100 on the nose. As the flat element 1 is made flat, it is possible to move the end parts 3 of the flat element 1 relative to each other thereby varying the distance between the end parts 3 for comfortable placement of the inhalation device 100 on the septum of the nose.

In an example embodiment, the flat element 1 may further include an adhesive layer 8 placed between the substrate 4 and the at least one layer 5 of the material impregnated with the volatile substances. The adhesive layer 8 may fix the layer 5 to the substrate 4. The adhesive layer 8 may include a glue.

The flat element 1 may be configured to be placed on the nose in such a way that the plane 6 (also referred to as a working plane) of the flat element 1 is located substantially perpendicularly to the septum of the nose and along the direction of a flow of exhaled air. Therefore, the inhalation device 100 does not impede free breathing because the plane 6 does not block airways of the patient.

The substrate 4 may have a bottom surface, an upper surface, and a lateral surface. The upper surface may accommodate the layer 5 of the material impregnated with volatile substances. The bottom surface and the lateral surface may face the skin of the patient, which provides heating of the substrate 4 together with the layer 5 on the substrate 4 and helps to accelerate the evaporation of the volatile substances from the layer 5. Furthermore, though the layer 5 does not contact the skin directly, the layer 5 may be located close to the skin and be heated by a heat exposed by the skin. When evaporating from the surface of the layer 5, the volatile substances enter the nasal passages of the patient, are carried by an inhaled air flow into respiratory channels and provide a beneficial effect to the respiratory system of the patient.

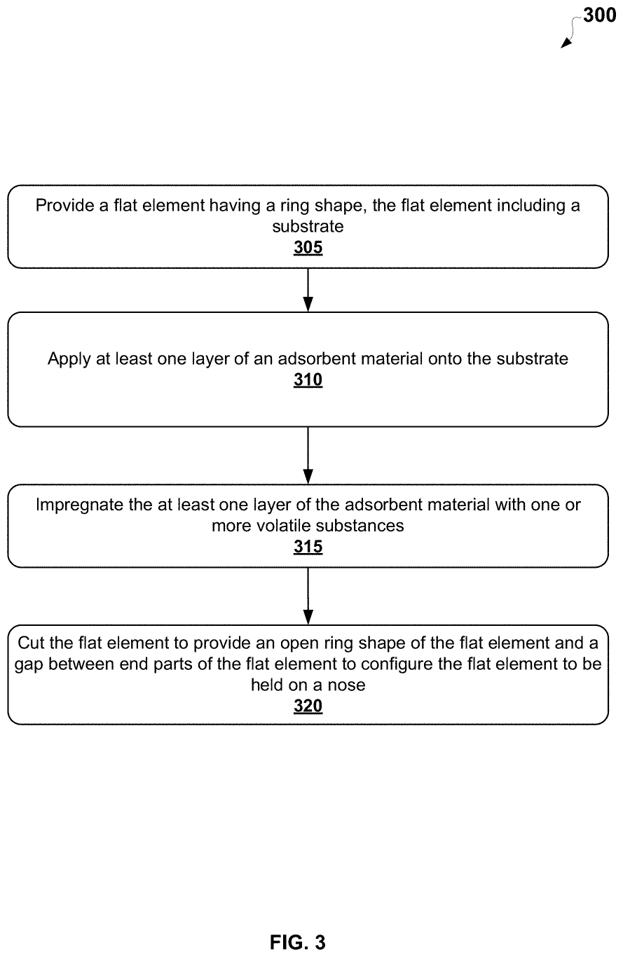

FIG. 3 is a flow chart illustrating a method 300 for providing an inhalation device, in accordance with an example embodiment. The method 300 may commence with providing a flat element having a ring shape at operation 305. The flat element may include a substrate. The substrate may be made of paper or cardboard.

The method 300 may include applying at least one layer of an adsorbent material onto the substrate at operation 310. The at least one layer of the adsorbent material may be made of a porous material or a material having a fiber structure. The adsorbent material is selected from the following materials: a porous polyurethane, cork tree, cotton, linen, paper, cardboard, wood veneer, silk, wool, felt, and combinations of these materials. The absorbent material can be placed directly on the substrate and attached to the substrate, e.g., by pressing.

The method 300 may further include impregnating the at least one layer of the adsorbent material with one or more volatile substances at operation 315. The one or more volatile substances may include at least one of essential oils, essential compositions, medicinal substances, and the like. In an example embodiment, the one or more volatile substances may be deposited onto the at least one layer of the adsorbent material by ultrasonic spraying of the one or more volatile substances onto the surface of the adsorbent material.

The method 300 may include cutting the flat element at operation 320 to provide an open ring shape of the flat element and a gap between end parts of the flat element to configure the flat element to be held on a nose. In an example embodiment, the open ring shape may include one of the following shapes: a horseshoe shape, a round open ring shape, an oval open ring shape, an ellipsoid open ring shape, a U-shape, and the like. Holding of the flat element on the nose may include holding of the flat element on a septum or wings of the nose. The method 300 may further include rounding the end parts of the flat element.

The method 300 may further include applying an adhesive layer between the substrate and the at least one layer of the adsorbent material. The adhesive layer may fix the at least one layer of the adsorbent material to the substrate. In an example embodiment, the adhesive layer may include a glue. Upon applying the adhesive layer, the at least one layer of the adsorbent material and the substrate may be fastened to each other by pressing.

The method 300 may further include placing the inhalation device into a sealed package. In some example embodiment, a plurality of inhalation device (i.e., a set of inhalation devices) may be packed into a sealed package.

Thus, an inhalation device and a method for providing an inhalation device are described. Although embodiments have been described with reference to specific exemplary embodiments, it will be evident that various modifications and changes can be made to these exemplary embodiments without departing from the broader spirit and scope of the present application. Accordingly, the specification and drawings are to be regarded in an illustrative rather than a restrictive sense.

What is claimed is:

1. An inhalation device, the inhalation device including a flat element having an open ring shape, the flat element having end parts and a gap provided between the end parts to configure the flat element to be held on a nose, the flat element having a horizontal plane disposed perpendicularly to a symmetry plane of the flat element, wherein the flat element includes at least two layers:
a substrate; and
at least one layer of a material impregnated with one or more volatile substances, the at least one layer being fixed on the substrate.

2. The inhalation device of claim 1, wherein the open ring shape includes one of the following shapes: a horseshoe shape, a round open ring shape, an oval open ring shape, an ellipsoid open ring shape, and a U-shape.

3. The inhalation device of claim 1, wherein the holding of the flat element on the nose includes holding of the flat element on a septum or wings of the nose.

4. The inhalation device of claim 1, wherein the substrate is made of paper or cardboard.

5. The inhalation device of claim 1, wherein the at least one layer of the material impregnated with the one or more volatile substances is made of a porous material or a material having a fiber structure.

6. The inhalation device of claim 1, wherein the end parts are rounded.

7. The inhalation device of claim 1, further comprising an adhesive layer placed between the substrate and the at least one layer of the material impregnated with the one or more volatile substances, the adhesive layer fixing the at least one layer to the substrate.

8. The inhalation device of claim 1, wherein the material for being impregnated with the one or more volatile substances is selected from the following materials: a porous polyurethane, cork tree, cotton, linen, paper, cardboard, wood veneer, silk, wool, felt, and combinations thereof.

9. The inhalation device of claim 1, wherein the one or more volatile substances include at least one of essential oils, essential compositions, and medicinal substances.

10. A method for providing an inhalation device, the method comprising:
providing a flat element having a ring shape, the flat element including a substrate;
applying at least one layer of an adsorbent material onto the substrate;
impregnating the at least one layer of the adsorbent material with one or more volatile substances; and
cutting the flat element to provide an open ring shape of the flat element and a gap between end parts of the flat element to configure the flat element to be held on a nose.

11. The method of claim 10, wherein the open ring shape includes one of the following shapes: a horseshoe shape, a round open ring shape, an oval open ring shape, an ellipsoid open ring shape, and a U-shape.

12. The method of claim 10, wherein the holding of the flat element on the nose includes holding of the flat element on a septum or wings of the nose.

13. The method of claim 10, wherein the substrate is made of paper or cardboard.

14. The method of claim 10, wherein the at least one layer of the adsorbent material is made of a porous material or a material having a fiber structure.

15. The method of claim 10, further comprising rounding the end parts of the flat element.

16. The method of claim 10, further comprising applying an adhesive layer between the substrate and the at least one layer of the adsorbent material, the adhesive layer fixing the at least one layer of the adsorbent material to the substrate.

17. The method of claim 10, wherein the adsorbent material is selected from the following materials: a porous polyurethane, cork tree, cotton, linen, paper, cardboard, wood veneer, silk, wool, felt, and combinations thereof.

18. The method of claim 10, wherein the one or more volatile substances include at least one of essential oils, essential compositions, and medicinal substances.

19. The method of claim 10, further comprising placing the inhalation device into a sealed package.

20. An inhalation device, the inhalation device including a flat element having an open ring shape, the flat element having end parts and a gap provided between the end parts to configure the flat element to be held on a nose, wherein the end parts are rounded, the flat element having a horizontal plane disposed perpendicularly to a symmetry plane of the flat element, wherein the flat element includes at least two layers:
a substrate;
at least one layer of a material impregnated with one or more volatile substances, the at least one layer being fixed on the substrate; and
an adhesive layer placed between the substrate and the at least one layer of the material impregnated with the one or more volatile substances, the adhesive layer fixing the at least one layer to the substrate.
